(19) United States
(12) Patent Application Publication
(10) Pub. No.: US 2010/0121383 A1
(43) Pub. Date: May 13, 2010

(54) METHOD, SYSTEM, AND APPARATUS FOR MAMMALIAN BONY SEGMENT STABILIZATION

(76) Inventors: Todd Stanaford, Midland, TX (US); Leonel Dominguez, Jacksonville, FL (US)

Correspondence Address: MERLE W. RICHMAN, III, P.O. BOX 3333, LA JOLLA, CA, 92038, US

(21) Appl. No.: 12/268,402

(22) Filed: Nov. 10, 2008

(51) Int. Cl. A61B 17/70; A61B 19/00

(52) U.S. Cl. 606/280; 128/898; 606/286; 606/70

(57) ABSTRACT

Embodiments of bony region stabilization are described generally herein. Other embodiments may be described and claimed.

### TECHNICAL FIELD

Various embodiments described herein relate generally to stabilizing mammalian bony segments, including systems and methods employing an elongated element to stabilize one or more mammalian bony segments.

### BACKGROUND INFORMATION

It may be desirable to stabilize one or more bony segments via an elongated element, the present invention provides such treatment.

### BRIEF DESCRIPTION OF THE DRAWINGS

FIG. 1 is a simplified diagram of mammalian bony segment stabilization architecture according to various embodiments.

FIG. 2A is a simplified, isometric front view of a mammalian bony segment stabilization system elongated element according to various embodiments.

FIG. 2B is a simplified, isometric rear view of a mammalian bony segment stabilization system elongated element according to various embodiments.

FIG. 2C is a simplified, isometric front, partial view of a mammalian bony segment stabilization system elongated element bony fixation element coupling segment according to various embodiments.

FIG. 2D is a simplified, isometric front, partial view of a mammalian bony segment stabilization system elongated element bony fixation element retention module cavity according to various embodiments.

FIG. 3 is a simplified, isometric diagram of a bony fixation element retention module according to various embodiments.

FIG. 4A is a simplified, partial front view of a mammalian bony segment stabilization system including an elongated element, a bony fixation element retention module, and a bony fixation element according to various embodiments.

FIG. 4B is a simplified, partial side view of a mammalian bony segment stabilization system including an elongated element, a bony fixation element retention module, and bony fixation element according to various embodiments.

FIG. 5 is a simplified, partial side view of a mammalian bony segment stabilization system including an elongated element, bony fixation element retention module, a bony fixation element, and a bony fixation element retention module deflection pin according to various embodiments.

FIG. 6A is a simplified, partial side view of a mammalian bony segment stabilization system including an elongated element, a bony fixation element retention module, a bony fixation element, a bony fixation element retention module deflection pin, and a fixation element driver according to various embodiments.

FIG. 6B is a simplified, full side view of a mammalian bony segment stabilization system including an elongated element, bony fixation element retention module, bony fixation element, a bony fixation element retention module deflection pin, and a fixation element driver according to various embodiments.

FIG. 7 is a simplified, full side isometric view of a mammalian bony segment stabilization system fixation element driver according to various embodiments.

FIG. 8 is a simplified, full side isometric view of a mammalian bony segment stabilization system bony fixation element retention module deflection pin tool according to various embodiments.

FIG. 9A-9B are flow diagrams illustrating mammalian bony segment stabilization processing algorithms according to various embodiments.

### DETAILED DESCRIPTION

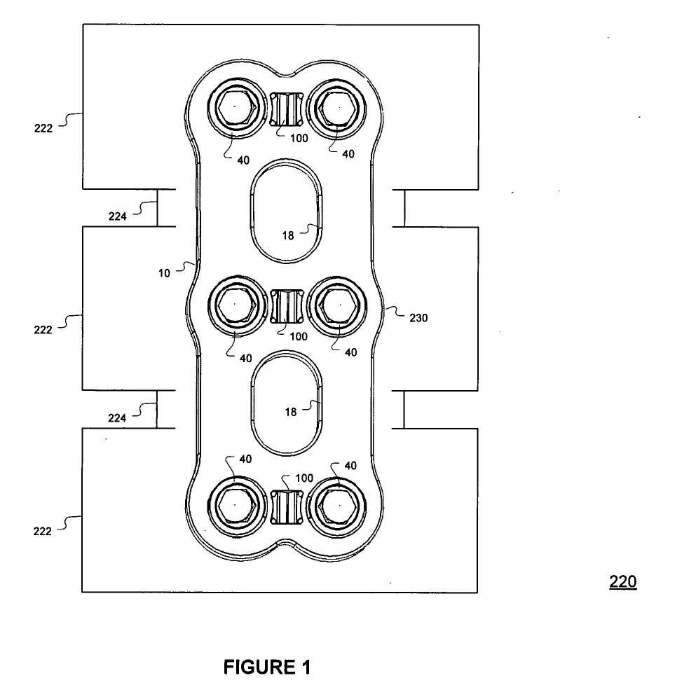

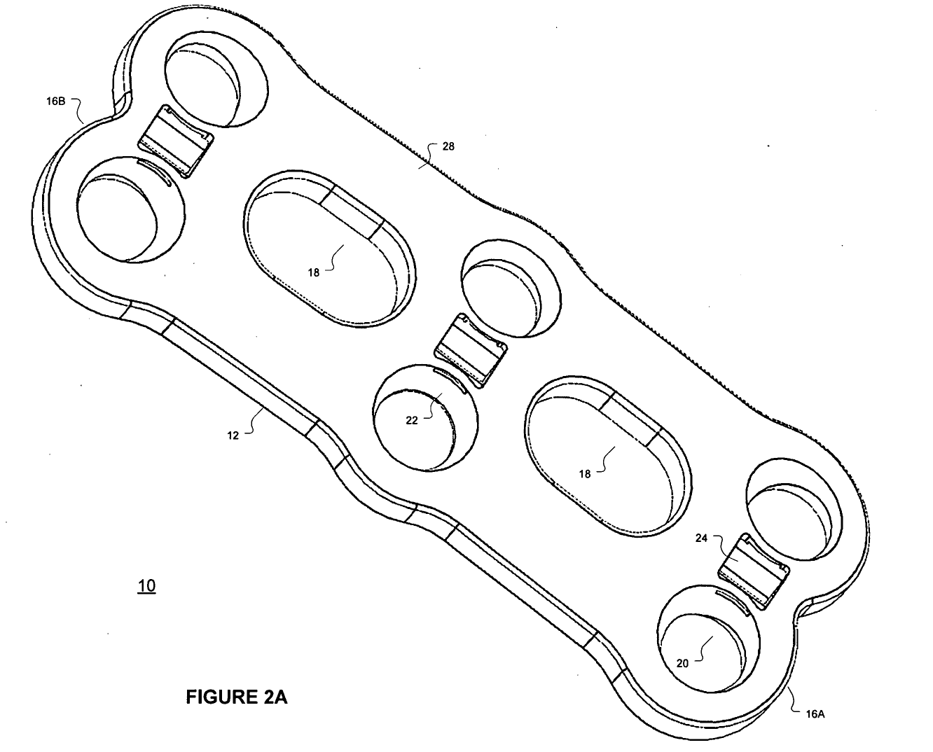

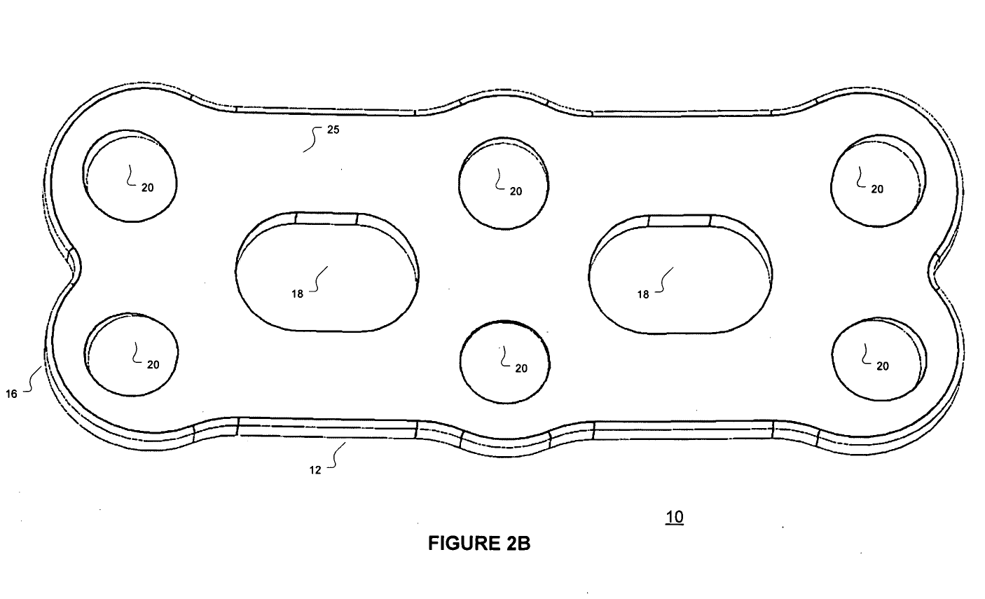

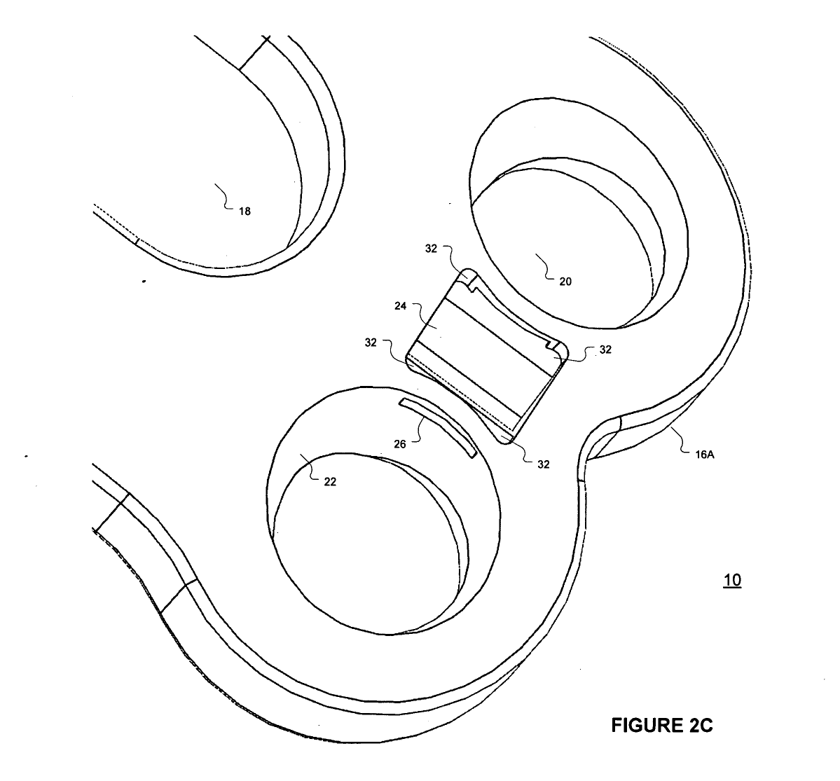

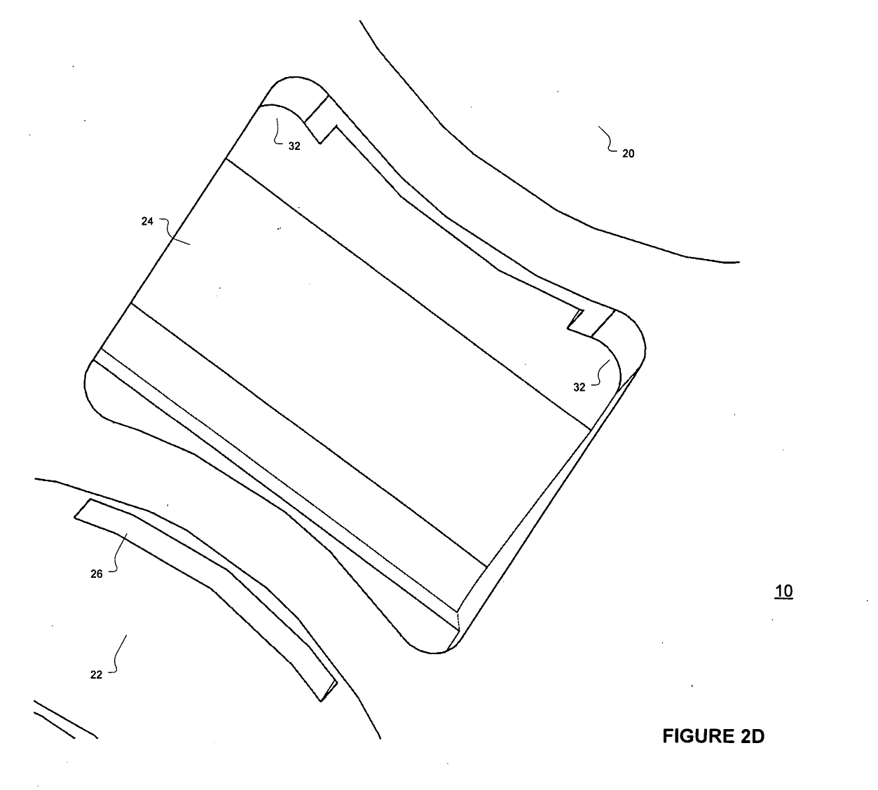

FIG. 1 is a simplified diagram of a mammalian bony segment stabilization architecture 220 according to various embodiments. The architecture 220 includes a mammalian bony segment stabilization system 230 coupled to a plurality of bony regions 222. The bony segment stabilization system 230 includes an elongated element 10 extending to at least two or more bony regions 222 to be stabilized. The elongated element 10 may include at least one opening (20 in FIG. 2A) adjacent or within the two or more bony regions 222. The elongated element 10 may be fixably coupled to each bony region 222 via the opening 20 and a bony coupling element 40 such as a screw, pin, or other bony region coupling or fixation element.

In the embodiment 230 the elongated element 10 includes two openings 20 that may accommodate at least one bony fixation element 40. The system 230 couples three bony regions 222 in an each embodiment and includes six openings 20 and six corresponding spinal fixation elements 40. The elongated element 10 may also include an opening 18 that may enable a user to insert or visualize implants in the region 224 and visually inspect the region 224. The implants may be comprised of any biocompatible material including bone, polymers, and metals. Further the elongated element 10 may be comprised of any biocompatible material including bone, polymers, and metals.

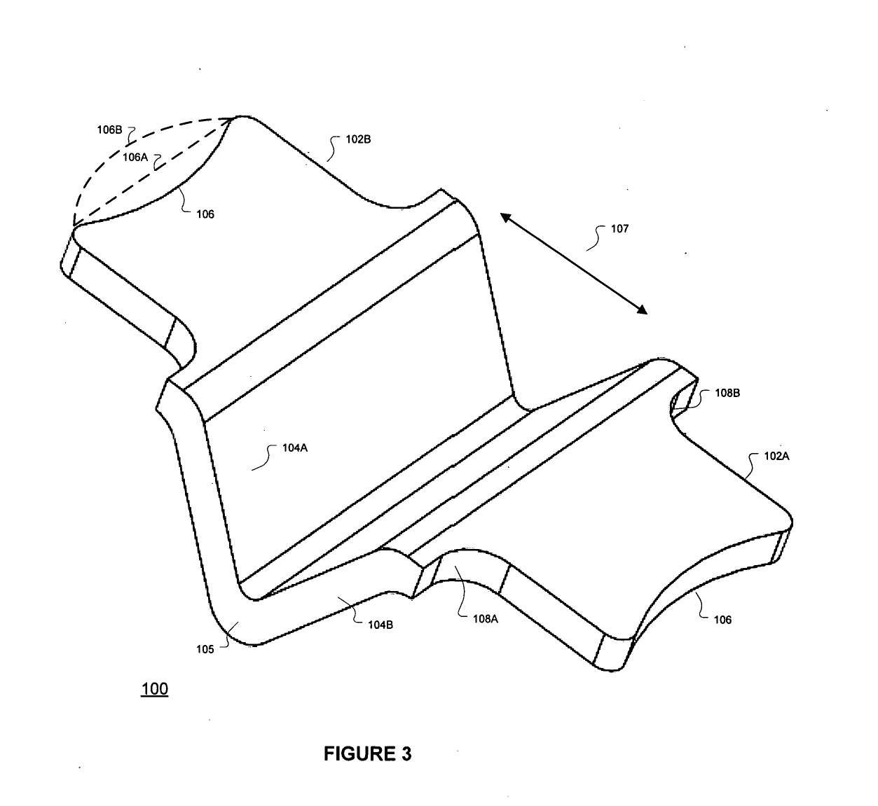

In an embodiment the bony segment stabilization system 230 further includes at least one bony fixation element retention module 100. The bony fixation element retention module 100 may engage a bony fixation element 40 via a slot 26 in an opening 20 (FIG. 2C). The bony fixation element retention module 100 may include at least one arm 106 (FIG. 3) where the arm 106 may limit or prevent bony fixation element 40 dislocation from a bony region 222 and may limit or prevent bony fixation element 40 projection beyond a front surface 28 (FIG. 2A) of the elongated element 10.

In an embodiment one or more bony regions 222 may be separated by one or more non-bony elements 224, for example bony regions 222 may be vertebra separated by spinal discs 224 in a cervical, thoracic, or lumbar region of a mammal including a human. In another embodiment the bony regions 222 may be part of a single, fractured bone to be stabilized such a femur or other long mammalian bone.

FIG. 2A is a simplified, isometric front view and FIG. 2B is a simplified, isometric rear view of the mammalian bony segment stabilization system elongated element 10 according to various embodiments. FIG. 2C is a simplified, isometric front, partial view of a mammalian bony segment stabilization system elongated element bony fixation element coupling segment according to various embodiments. FIG. 2D is a simplified, isometric front, partial view of a mammalian bony segment stabilization system elongated element bony fixation element retention module cavity according to various embodiments. The elongated element 10 includes several bony fixation element openings 20, implant and region openings 18, and at least one bony fixation element retention cavity 24. The elongated element 10 includes a front side 28, rear side 25, side portions 12, a top portion 16B, and a bottom portion 16A.

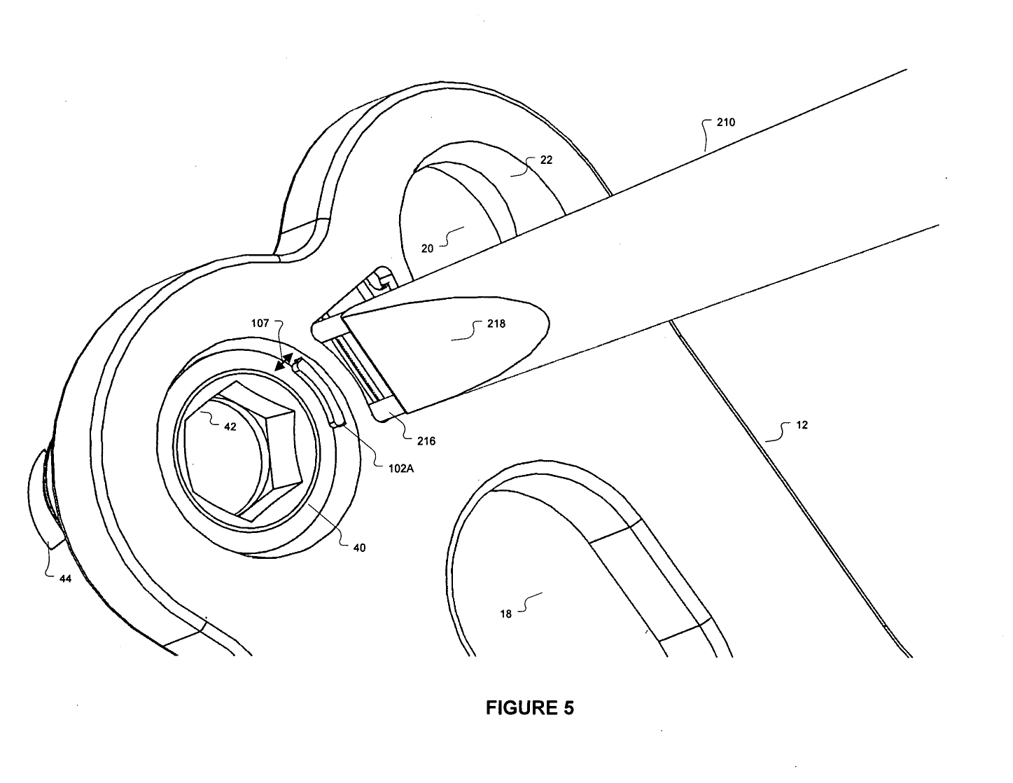

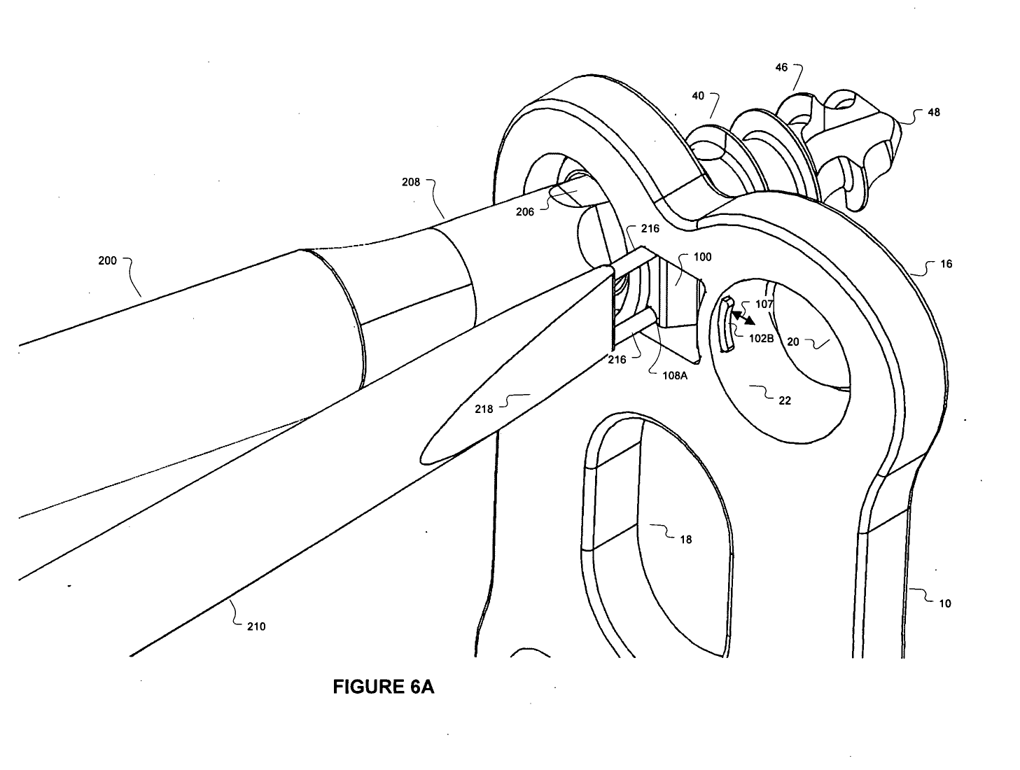

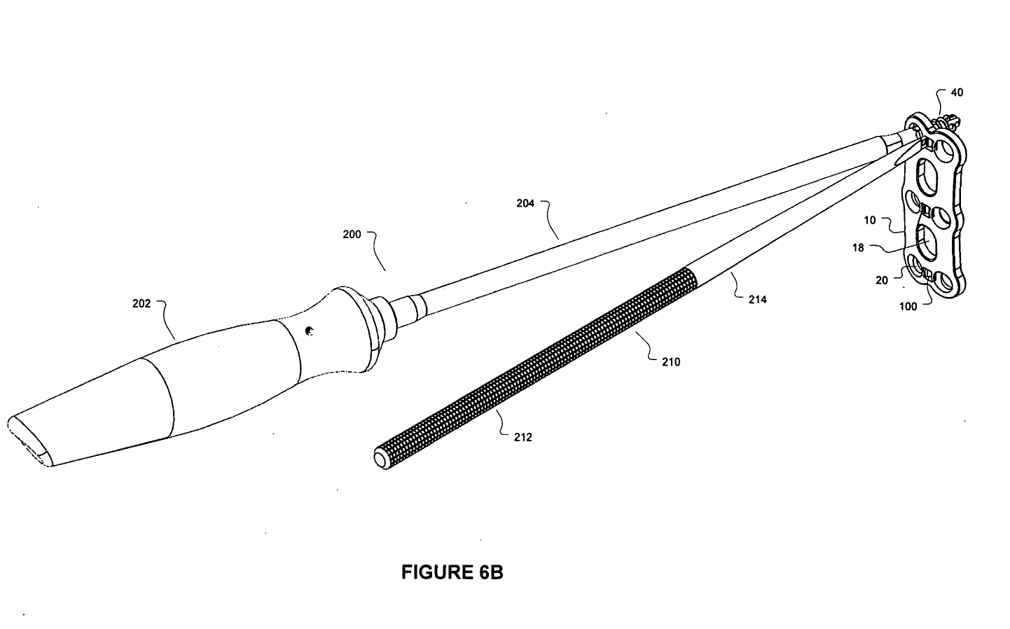

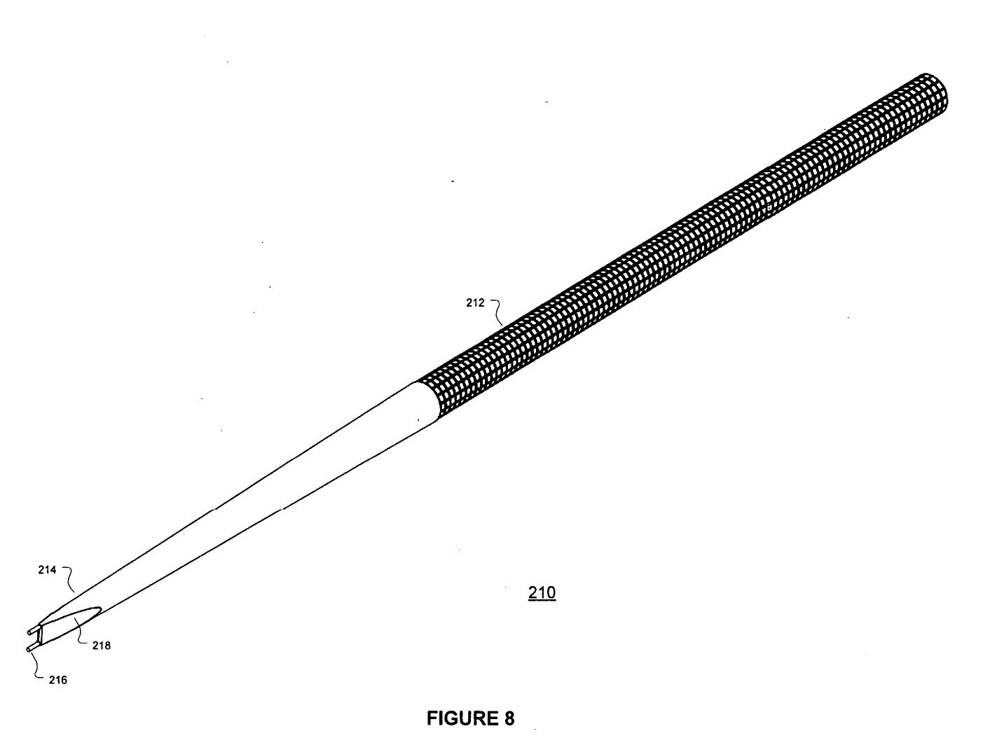

In an embodiment the cavity 24 may be configured to hold a bony fixation element retention module (100 in FIG. 3) in a robustly deformable manner. In an embodiment each bony fixation element opening 20 may have a sloped or partially spherical wall 22 that may engage an inverse or complementary shape of a bony fixation element 40 head 42. The opening 20 may include a slot 26 coupling the bony fixation element retention module cavity 24 to the opening 20. The module cavity 24 may be recessed with a configuration/shape complementary to the bottom of the bony fixation element retention module 100. The retention module cavity 24 may further include retention module deflection pin access points or sites 32 in one or more corners. One or more pins 216 of a bony fixation element retention module deflection tool 210 (FIG. 8) may be insertable into the retention module deflection pin access points 32 as shown in FIGS. 5, 6A, and 6B.

FIG. 3 is a simplified, isometric diagram of a bony fixation element retention module 100 according to various embodiments. The module 100 includes a central axis 105 with two deformable arms 104A, 104B each coupled to bony fixation element retention arm 102A, 102B. Each bony fixation element retention arm 102A, 102B may include a convex 106, flat 106A, or concave 106B end. In an embodiment a bony fixation element retention arm 102A or 102B is extended from the cavity 24 through a first slot 26 and the end 106 of the arm 102A, 102B extends into the opening 20. The other bony fixation element retention arm 102A or 102B is extended from the cavity 24 through the opposite slot 26 and the end 106 of the other arm 102A, 102B extends into an opposite opening 20. The retention module 100 may include insets 108A, 108B where the insets may be engaged by a pin 216 of a bony fixation element retention module deflection tool 210 (FIG. 8). The arms 102A, 102B may move restoratively toward the central axis 105 in the direction shown by 107.

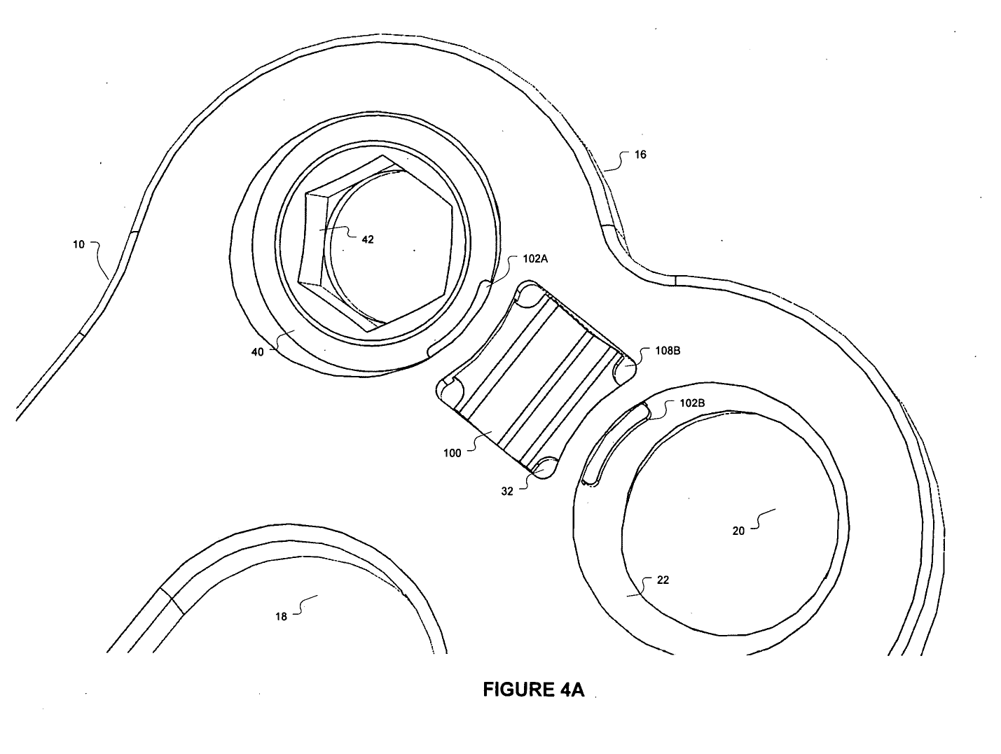

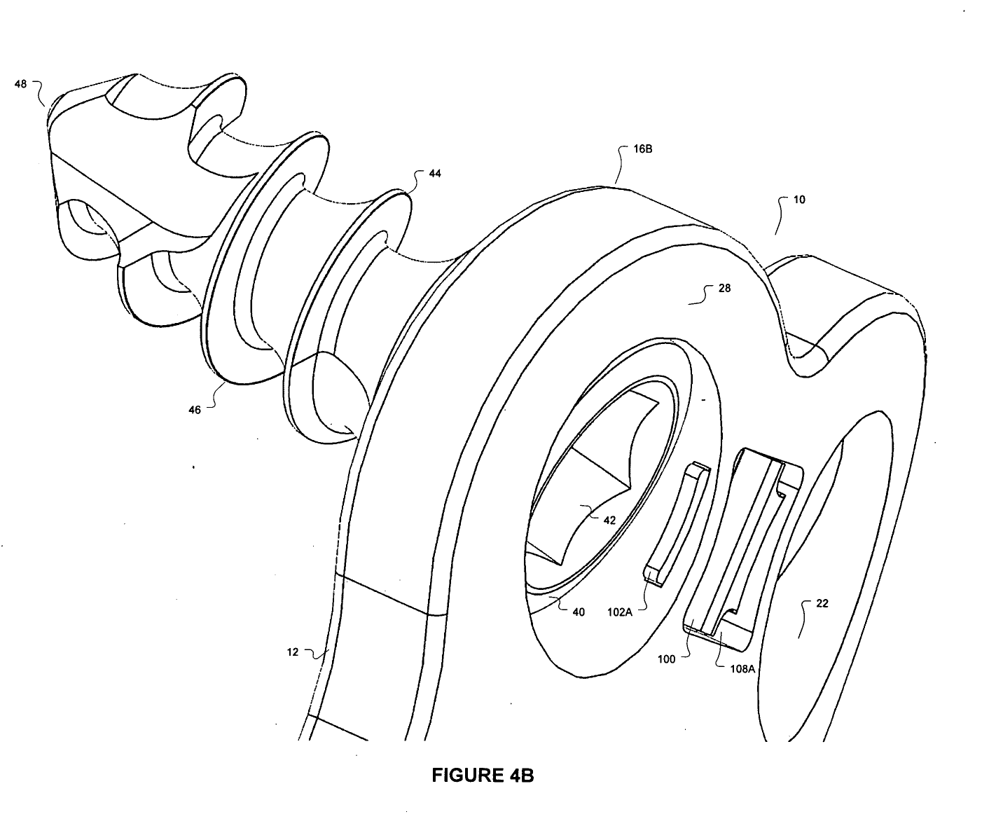

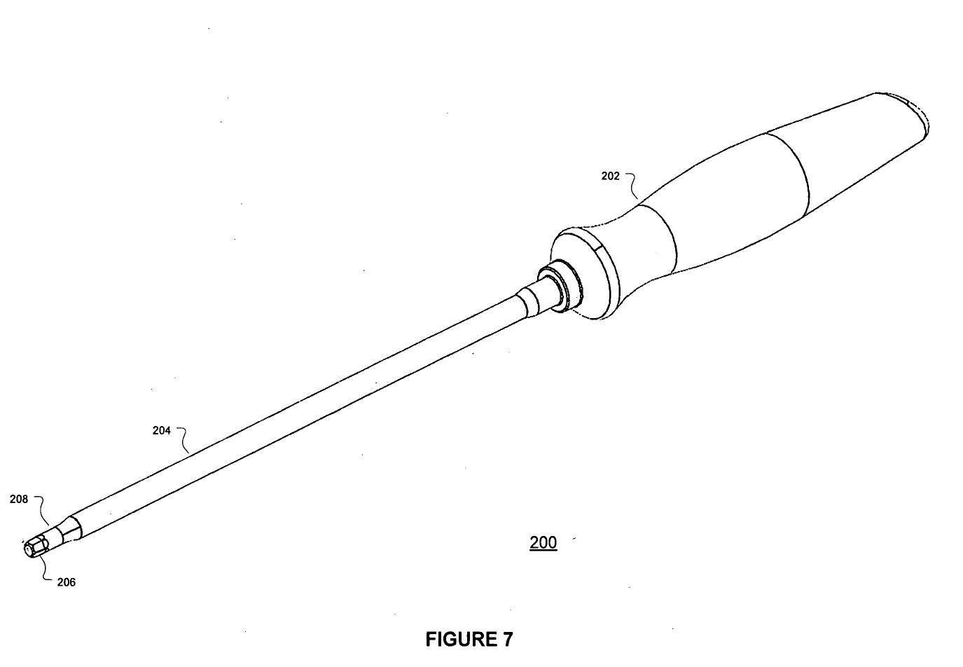

FIG. 4A is a simplified, partial front view and FIG. 4B is a simplified, partial side view of a mammalian bony segment stabilization system 230 including an elongated element 10, a bony fixation element retention module 100, and bony fixation element 40 according to various embodiments. As shown in FIGS. 4A and 4B the retention module 100 arms 102A and 102B extend into openings 20 and prevent or limit the movement of the bony fixation element 40 and may limit or prevent the bony fixation element 40 head 42 from extending beyond the elongated element 10 front section 28. The bony fixation element 40 may include a head 42 and a shaft 44. In an embodiment the shaft 44 may include a tip 48 and a thread 46. The head 42 may include a tool compatible recess, in an embodiment a female hexagonal recess for a male hexagonal tool (200 of FIG. 7 with male hexagonal tip 206.)

FIG. 5 is a simplified, partial side view of a mammalian bony segment stabilization system 230 including an elongated element 10, a bony fixation element retention module 100, a bony fixation element 40, and a bony fixation element retention module deflection pin tool 210 according to various embodiments. In to remove the bony fixation element 40, the retention module 100 arm 102A may be reversibly deflected from the opening 20 toward the module 100 center axis 105. The bony fixation element may then be removed. The retention module 100 arm 102A may be reversibly deflected from the opening 20 toward the module 100 center axis 105 in order to employ a bony fixation element 40 within a bony region through the opening 20.

FIG. 6A is a simplified, partial side view and FIG. 6B is a simplified, full side view of a mammalian bony segment stabilization system 230 including an elongated element 10, a bony fixation element retention module 100, a bony fixation element 40, a bony fixation element retention module deflection pin tool 210, and a bony fixation element driver 200 according to various embodiments. As shown in FIGS. 6A and 6B, pins 216 of pin tool 210 may engage the recesses 108A of the retention module 100 via the pin access points 32 of the elongated element 10. A bony fixation element driver 200 may include a male head 206 that may engage a female recess of the bony fixation element 40. The retention module 100 arm 102A may be reversibly deflected from the opening 20 toward the module 100 center axis 105 via the pins 216 of the pin tool 210 in order to operate the driver 200 to either insert or remove a bony fixation element 40 in or from within a bony region 222 through the opening 20.

FIG. 7 is a simplified, full side isometric view of a mammalian bony segment stabilization system fixation element driver 200 according to various embodiments. The driver 200 includes a handle 202, shaft 204, and tip 208. In an embodiment the tip 208 includes a male hexagonal head 206. The driver 200 may be used to engage a bony fixation element female recess 42 in order to operate the bony fixation element 40. One or more bony fixation elements 40 may be employed to couple the elongated element 10 to one or more bony regions 222.

FIG. 8 is a simplified, full side isometric view of a mammalian bony segment stabilization system bony fixation element retention module deflection pin tool 210 according to various embodiments. The pin tool 210 may include a handle 212, shaft 214, and a slanted tip 218. The slanted tip 218 may include one or more deflection pins 216. As noted, the retention module 100 arm 102A may be reversibly deflected from the opening 20 toward the module 100 center axis 105 via the pins 216 of the pin tool 210 in order to operate the driver 200 to either insert or remove a bony fixation element 40 in or from within a bony region 222 through the opening 20.

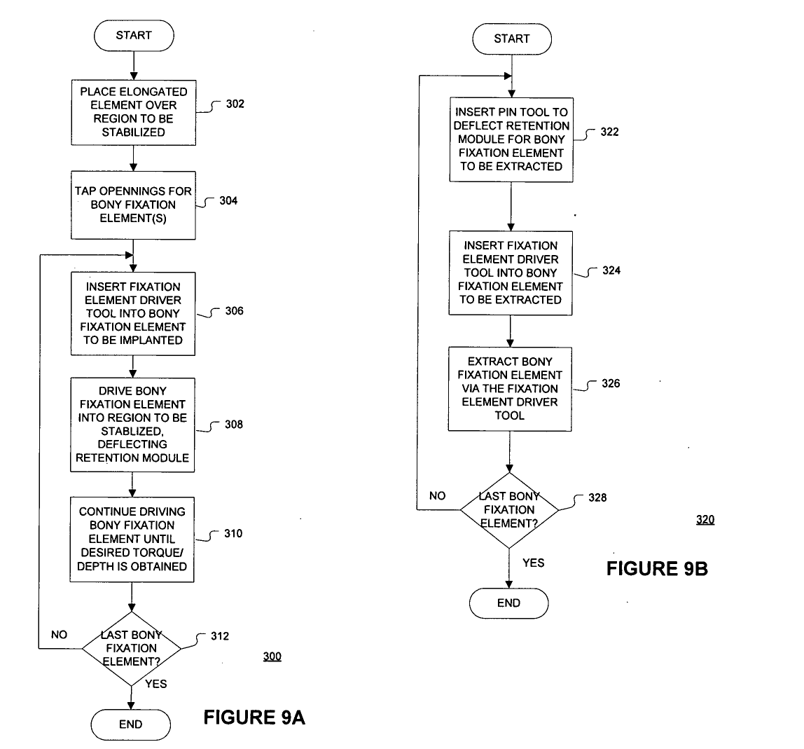

FIG. 9A-9B are flow diagrams illustrating mammalian bony segment stabilization processing algorithms 300 and 320 according to various embodiments. The algorithm 300 may be employed to stabilize one or more bony regions 222. A user such as a surgeon may place an elongated element 10 having a plurality of openings 20 over or adjacent to at least one region 222 to be stabilized (activity 302). A user may then create or tap openings in the region 222 corresponding to one or more openings 20 of the elongated element 10. The tapped openings may correspond to the bony fixation element(s) to be insert through the elongated element 10 openings 20 to stabilize the region(s) 222 (activity 304).

A user may then insert the bony fixation driver tool 200 tip 208 into a corresponding segment a bony fixation element 40 where the bony fixation element 40 is to be inserted into the tapped opening (activity 306). A user may then drive the bony fixation element 40 through an opening 20 of the elongated element 10 into bony region(s) 222 to be stabilized (activity 308), thereby deflecting an arm 102A, 102B of the retention module 100. A user may continue driving the bony fixation element 40 through the opening 20 of the elongated element 10 into bony region(s) 222 to be stabilized (activity 308), while deflecting the arm 102A, 102B of the retention module 100 until a desired torque is obtained (activity 310) or the bony fixation element 40 head 42 lies below the elongated element 10 face 28. A user may repeat activities 306, 308, 310 until all the desired bony fixation elements 40 have been inserted (activity 312).

The algorithm 320 may be employed to remove one or more bony fixation elements 40 of a mammalian bony segment stabilization system 230. A user may then insert one or more pins 216 of a bony retention module deflection tool 210 into a corresponding recess 32 of an elongated element 10 and adjacent a recess 108A, 108B of a retention module 100 where the retention module 100 arms 102A, 102B is located in the elongated element 10 opening 20 occupied by the bony fixation element 40 to be removed (activity 322). A user may then insert the bony fixation driver tool 200 tip 208 into a corresponding segment a bony fixation element 40 where the bony fixation element 40 is to be removed from the elongated element 10 opening 20 (activity 324). A user may extract the bony fixation element 40 through the opening 20 of the elongated element 10 from a bony region(s) 222 while the arm 102A, 102B of the retention module 100 is deflected by the deflection tool 210 until the bony fixation element is extracted (activity 326). A user may repeat activities 322, 324, 326 until all the desired bony fixation elements 40 have been extracted (activity 320).

The accompanying drawings that form a part hereof show, by way of illustration and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In the foregoing Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment. Thus the following claims are hereby incorporated into the Detailed Description, with each claim standing on its own as a separate embodiment.

What is claimed is:

1. A method for stabilizing a bony region, comprising:
fixably coupling an elongated element to the bony region with a first bony fixation element, the elongate element having a long axis, a front and a back, the elongate element including:
a first opening configured to receive the first bony fixation element and extending from the elongate element front to the elongate element back; and
a cavity within the elongated element; and
securely seating a bony fixation element retention module within the elongated element cavity, the retention module including a first deflectable arm, the first deflectable arm extending into the first opening.

2. The method of claim 1, wherein the retention module first deflectable arm limits the protrusion of the first bony fixation element in the first opening beyond the elongated element front.

3. The method of claim 2, the elongate element further comprising a first slot communicating between the first opening and the cavity.

4. The method of claim 3, the retention module first deflectable arm extending from the cavity into the first opening via the first slot.

5. The method of claim 2, the elongated element further comprising a second opening configured to receive a second bony fixation element and extending from the elongate element front to the elongate element back.

6. The method of claim 5, further comprising:
fixably coupling an elongated element to the bony region with the second bony fixation element; and
the retention module including a second deflectable arm, the second deflectable arm extending into the second opening.

7. The method of claim 6, wherein the retention module second deflectable arm limits the protrusion of the second bony fixation element in the second opening beyond the elongated element front.

8. The method of claim 7, the second slot communicating between the second opening and the cavity.

9. The method of claim 8, the retention module second deflectable arm extending from the cavity into the second opening via the second slot.

10. The method of claim 9, wherein the bony region includes a vertebral body.

11. The method of claim 1, deflecting the retention module first deflectable arm toward the cavity to enable removal of the first bony fixation element from the first opening beyond the elongated element front.

12. An apparatus for stabilizing a bony region, comprising:
an elongated element having a long axis, a front and a back, including:
a first opening configured to receive a first bony fixation element and extending from the elongate element front to the elongate element back; and
a cavity within the elongated element; and
a bony fixation element retention module configured to be securely seated within the elongated element cavity, the retention module including a first deflectable arm, the first deflectable arm extending into the first opening when the retention module is securely seated within the elongated element cavity.

13. The apparatus of claim 12, wherein the retention module first deflectable arm limits the protrusion of the first bony fixation element seated in the first opening beyond the elongated element front.

14. The apparatus of claim 13, further comprising a first slot communicating between the first opening and the cavity.

15. The apparatus of claim 14, the retention module first deflectable arm extending from the cavity into the first opening via the first slot.

16. The apparatus of claim 13, the elongated element further comprising a second opening configured to receive a second bony fixation element and extending from the elongate element front to the elongate element back and the retention module further comprising a second deflectable arm, the second deflectable arm extending into the second opening when the retention module is securely seated within the elongated element cavity.

17. The apparatus of claim 16, wherein the retention module second deflectable arm limits the protrusion of a second bony fixation element seated in the second opening beyond the elongated element front.

18. The apparatus of claim 17, further comprising a second slot communicating between the second opening and the cavity.

19. The apparatus of claim 18, the retention module second deflectable arm extending from the cavity into the second opening via the second slot.

20. The apparatus of claim 13, wherein the bony region includes a vertebral body.

21. The apparatus of claim 13, the elongated element further comprising:
a second opening configured to receive a second bony fixation element and extending from the elongate element front to the elongate element back; and
a second cavity within the elongated element.

22. The apparatus of claim 21, further comprising a second bony fixation element retention module configured to securely seated within the second elongated element cavity, the second retention module including a first deflectable arm, the first deflectable arm extending into the second opening when the second retention module is securely seated within the second elongated element cavity.

23. The apparatus of claim 22, wherein the second retention module first deflectable arm limits the protrusion of a second bony fixation element seated in the second opening beyond the elongated element front.

24. The apparatus of claim 23, further comprising a slot communicating between the second opening and the second cavity.

25. The apparatus of claim 24, the second retention module deflectable arm extending from the second cavity into the second opening via the slot.

26. The apparatus of claim 25, the first opening couplable to a first region and the second opening couplable to a second region.

27. The apparatus of claim 26, the first opening couplable to a first vertebra and the second opening couplable to a second, adjacent vertebra.

28. The method of claim 12, wherein the retention module first deflectable arm is deflectable toward the cavity to enable removal of the first bony fixation element from the first opening beyond the elongated element front.
